(19) United States
(12) Patent Application Publication
(10) Pub. No.: US 2010/0143420 A1
(43) Pub. Date: Jun. 10, 2010

(54) MULTI-PHASIC PHARMACEUTICAL FORMULATIONS OF POORLY WATER-SOLUBLE DRUGS FOR REDUCED FED/FASTED VARIABILITY AND IMPROVED ORAL BIOAVAILABILITY

(76) Inventors: Dinesh Shenoy, Karnataka State (IN); Robert Lee, Boyertown, PA (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/523,445

(22) PCT Filed: Jan. 22, 2008
(86) PCT No.: PCT/US08/51639

§ 371 Date: Feb. 12, 2010

Related U.S. Application Data
(60) Provisional application No. 60/881,470, filed on Jan. 22, 2007.

(51) Int. Cl. A61K 38/12; A61P 1/08; A61P 9/00; A61P 25/00; A61P 21/02; A61P 7/02; A61K 31/216; A61K 31/436; A61K 31/58; A61K 31/192; A61K 31/496; A61K 31/343; A61K 31/5377; A61K 9/14

(52) U.S. Cl. 424/400; 514/543; 514/11; 514/291; 514/176; 514/569; 514/252.16; 514/462; 514/233.5

(57) ABSTRACT

Pharmaceutical formulations are disclosed comprising a multi-phasic pharmaceutical composition comprising an active pharmaceutical ingredient, wherein the active pharmaceutical ingredient is in a particulate state, a solubilized state, or in both a particulate state and in a solubilized state; a solvent; a non-miscible liquid; a stabilizer, and water; wherein the pharmaceutical formulation is an oral dosage form. Such pharmaceutical formulations are capable of reducing the fed/fast variability and improving oral bioavailability to which a number of active pharmaceutical ingredients are susceptible. The pharmaceutical formulations of the invention, therefore are bioequivalent in fed and fasted states and improved oral bioavailability.

### CROSS-REFERENCE TO RELATED APPLICATIONS

This application claims the benefit of U.S. Provisional Patent Application Ser. No. 60/881,470, entitled “Multi-phasic Pharmaceutical Formulations of Poorly Water-soluble Drugs for Reduced Fed/Fasted Variability and Improved Oral Bioavailability”, filed Jan. 22, 2007, the content of which is herein incorporated by reference in their entirety for all purposes.

### FIELD OF THE INVENTION

In general the invention is directed to multi-phasic compositions of poorly water-soluble drugs and methods of making the same. More particularly, these compositions may be used to reduce fed-fasted absorption variability and improve oral bioavailability of active pharmaceutical ingredients.

### BACKGROUND

An active pharmaceutical ingredient (API) that is readily soluble in water is not difficult to formulate into a suitable dosage form. Further, these drugs are unlikely to pose significant problems with respect to bioavailability when administered through oral or other routes of administration. However, formulating poorly water-soluble therapeutic drugs into suitable dosage forms poses a significant challenge. This is due to difficulties in making the API bioavailable in aqueous biological systems. In the case of formulations intended for oral administration, many poorly water-soluble APIs are susceptible to inadequate drug absorption, or are absorbed under wildly variable rates and/or extent of drug absorption. This can be further complicated by variability due to the presence or absence of food in the stomach; i.e., fed/fasted variability. Some poorly water-soluble APIs are never commercialized because they cannot be effectively solubilized in the biologic milieu, and therefore fail to exhibit acceptable in vivo therapeutic activity. Alternatively, the quantity of poorly water-soluble API required to be administered to achieve an acceptable level of therapeutic activity may be unreasonably high, given the poor water solubility of the agent, and possibly resulting in unacceptable toxicity.

Reduction of the particle size of the API results in increased surface area, which may result in greater water solubility and/or better dissolution properties. Techniques such as microprecipitation, micronization, milling, homogenization, super critical fluid particle generation, etc. have been used to reduce particle sizes of APIs. Exemplary milling techniques typically include dry and wet milling. However, dry milling does not offer additional benefits such as surface stabilization, increased wettability, or improved dispersion properties, and wet milling can be cost prohibitive for a number of APIs. There are several commercial technologies to address these issues namely NanoCrystal® technology from élan, NanoEdge® technology from Baxter, Insoluble Drug Delivery (IDD®) Technology from Skye Pharma, and Biorise® technology from Eurand.

Liquid form drug compositions are ubiquitous throughout the pharmaceutical industry, existing as compositions of solutions, suspensions, emulsions, and the like. While liquid dosage forms are convenient forms, especially for pediatric and geriatric applications, conversion of these liquid compositions to a solid dosage form (i.e., tablets or capsules) can add significantly to both patient compliance and the commercial value to the products. Simple aqueous-based solutions or suspensions may be converted to a corresponding solid dosage form by, for example, lyophilizing with suitable cryoprotectants, the resulting mass being mixed with one or more suitable diluents, followed by filling into capsules or compressing into tablets.

There is a need in the art for cost-effective methods of formulating poorly water-soluble and water-soluble API into suitable dosage forms exhibiting optimal in vivo efficacy. Particularly, there is a need for oral dosage forms of poorly water-soluble APIs which exhibit reduced fed/fasted absorption variability, or similar or bioequivalent absorption profiles when administered under fed and fasting conditions.

### SUMMARY

In one aspect, a pharmaceutical formulation is provided comprising a multi-phasic pharmaceutical composition in a dosage form, such as an oral dosage form, comprising: (a) an active pharmaceutical ingredient, wherein the active pharmaceutical ingredient is in a particulate state, a solubilized state, or in both a particulate state and in a solubilized state; (b) a solvent, (c) a non-miscible liquid, (d) a stabilizer, and (e) water.

Many prior art drug formulations exhibit a significant difference between the quantity of drug absorbed (AUC) and/or the rate of drug absorption (Cₘₐₓ and/or Tₘₐₓ) when the formulation is administered under fasting conditions as compared to when administered under fed conditions Preferably, the pharmaceutical formulations of the invention exhibit a reduced difference between the quantity of drug absorbed (AUC) and/or the rate of drug absorption (Cmax and/or Tmax) when the formulation is administered under fasting conditions as compared to when administered under fed conditions, as compared to a prior art formulation of the same API.

In one embodiment of the invention, upon administration of a pharmaceutical formulation of the invention to a mammal, the formulation exhibits an absorption profile (e.g., Cₘₐₓ and AUC, or Cₘₐₓ, AUC, and Tₘₐₓ) under fed conditions which is similar, or bioequivalent to, the absorption profile of the same composition administered under fasting conditions. In some embodiments, the mammal is a human.

In some embodiments, when a pharmaceutical formulation of the invention is administered to a rat or a rat model, the difference between the mean AUC determined at a fed state and the mean AUC determined at a fasted state is less than about 90,000 h*ng/ml. In other embodiments, the difference between the mean AUC determined at a fed state and the mean AUC determined at a fasted state is selected from the group consisting of less than about 85,000 h*ng/ml, less than about 80,000 h*ng/ml, less than about 75,000 h*ng/ml, less than about 70,000 h*ng/ml, less than about 65,000 h*ng/ml, less than about 60,000 h*ng/ml, less than about 55,000 h*ng/ml, less than about 50,000 h*ng/ml, less than about 45,000 h*ng/ml, less than about 40,000 h*ng/ml, less than about 35,000 h*ng/ml, less than about 30,000 h*ng/ml, less than about 25,000 h*ng/ml, less than about 20,000 h*ng/ml, less than about 15,000 h*ng/ml, and less than about 10,000 h*ng/ml.

In other embodiments, upon administration of a pharmaceutical formulation of the invention to a mammal, the percent difference between the mean AUC, mean Cₘₐₓ, and/or mean Tₘₐₓ determined at a fasted state and the mean AUC, mean Cₘₐₓ, and/or mean Tₘₐₓ determined at a fed state is less than about 1000%, less than about 900%, less than about 800%, less than about 700%, less than about 600%, less than about 500%, less than about 400%, less than about 300%, less than about 200%, less than about 100%, less than about 90%, less than about 80%, less than about 70%, less than about 60%, less than about 50%, less than about 40%, less than about 30%, less than about 20%, less than about 10%, less than about 9%, less than about 8%, less than about 7%, less than about 6%, less than about 5%, less than about 4%, less than about 3%, less than about 2%, less than about 1%, less than about 0.5%, or less than about 0.1%

In one embodiment, upon administration of a pharmaceutical formulation of the invention to a mammal, the formulation exhibits a difference in the relative exposure of the API between a fed and a fasted state of less than about 1000%. In other embodiments, the difference in the relative exposure between a fed and a fasted state is selected from the group consisting of less than about less than about 900%, less than about 800%, less than about 700%, less than about 600%, less than about 500%, less than about 400%, less than about 300%, less than about 200%, less than about 100%, less than about 90%, less than about 80%, less than about 70%, less than about 60%, less than about 50%, less than about 45%, less than about 40%, less than about 35%, less than about 30%, less than about 25%, less than about 20%, less than about 15%, less than about 10%, less than about 5%, less than about 4%, and less than about 3%.

In some embodiments, the oral dosage form the pharmaceutical formulations is a solid dosage form, and in other embodiments it is a liquid dosage form. The dosage form can be for oral administration, or for any other pharmaceutically acceptable method of administration. The compositions of the invention may also be potentially used for other routes of administration (topical, transdermal, vaginal, rectal, nasal, ocular, ungual, parenteral, mucosal etc.) and offer similar benefits of improving bioavailability of the drug.

In some embodiments, the API is fenofibrate, cyclosporine, sirolimus, danazol, naproxen, sildenafil, griseofulvin, mycophenolate or mofetil, or a mixture of any two or more thereof.

Methods are also provided for preparing the pharmaceutical formulations described herein. Thus, in another aspect, methods of preparing the pharmaceutical formulations comprise mixing the API, the solvent, the stabilizer, and the non-miscible liquid to form a first mixture; emulsifying the first mixture with water to form a multi-phasic pharmaceutical composition; and formulating the multi-phasic pharmaceutical composition into a suitable dosage form, such as an oral dosage form. In embodiments where the dosage form is a solid dosage form, the method can further comprise mixing the emulsified first mixture with an adsorbent carrier.

In addition, in one embodiment of the invention, the compositions of the invention may further comprise suitable inactive ingredients, including but not limited to viscosity modifiers, coloring and flavoring agents, etc.

The compositions and methods of the invention also may facilitate compounding and administration of more than one drug.

Both the foregoing general description and the following detailed description are exemplary and explanatory and are intended to provide further explanation of the invention as claimed. Other objects, advantages, and novel features will be readily apparent to those skilled in the art from the following detailed description of the invention.

### BRIEF DESCRIPTION OF THE DRAWINGS

FIG. 1 is a particle size distribution of raw fenofibrate, with a mean particle size of 57 μm.

FIG. 2 is a particle size distribution of fenofibrate following particle size reduction using the methods of U.S. Provisional Patent Application No. 60/779,420, applicable to the present invention, with a mean fenofibrate nanoemulsion droplet size of 60 nm (within the emulsion droplets), and with 100% of the fenofibrate particles having a size of less than 3 μm.

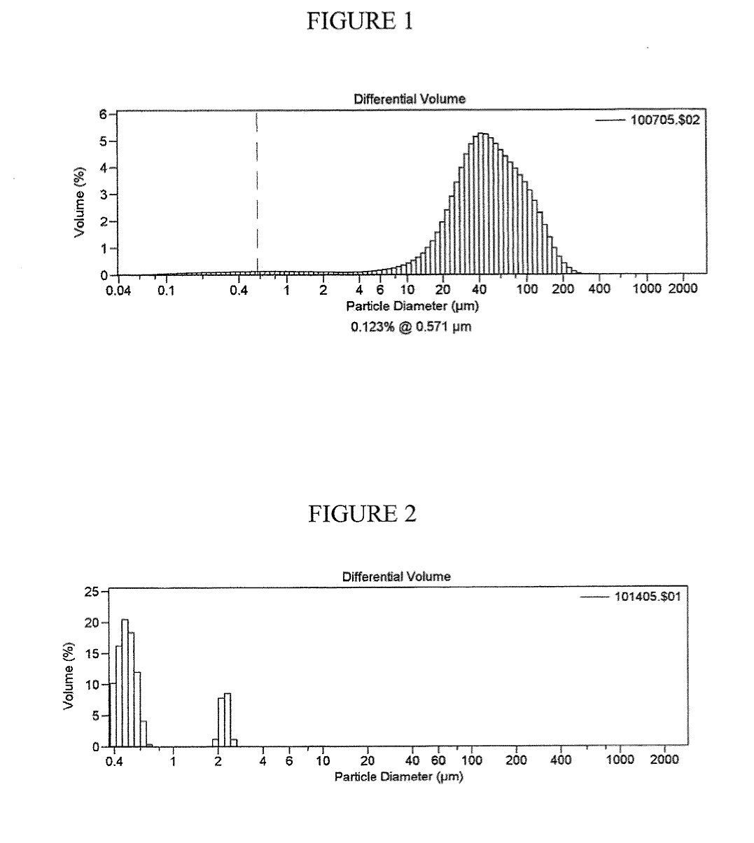

### DETAILED DESCRIPTION

### A. Definitions

The present invention is described herein using several definitions, as set forth below and throughout the application.

For the purposes of this disclosure and unless otherwise specified, “a” or “an” means “one or more.”

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

“Adsorbent carrier” refers to materials, usually solid, employed to adsorb and/or absorb a liquid formulation.

As used herein, the terms “capsules,” “tablets,” “lozenges,” and “cachets” are synonymous terms and are used interchangeably, any individual term representing the group, unless specifically noted that only a capsule, a tablet, a lozenge, or a cachet is envisioned for a particular purpose.

“API” is an abbreviation for active pharmaceutical ingredient.

“Cellulose” includes the various forms of cellulose known for use in pharmaceutical formulations, including but not limited to, ethyl cellulose, cellulose acetate, carboxymethyl cellulose calcium, sodium carboxymethyl cellulose, methylcellulose, hydroxyethyl cellulose, hydroxypropyl cellulose, hydroxypropyl methylcellulose, Hydroxypropyl methylcellulose phthalate, microcrystalline cellulose, and mixtures thereof.

Croscarmellose sodium is cross-linked sodium carboxymethyl cellulose.

“Crospovidone” is a water-insoluble cross-linked homopolymer of 1-vinyl-2-pyrrolidinone.

“Cyclodextrin” refers to a family of cyclic oligosaccharides containing at least six D-(+)-glucopyranose units.

“Emulsifier,” as used herein, refers to a material that promotes the formation of an emulsion.

As used herein, the term “emulsion” refers to a dispersion of one non-miscible liquid in another liquid.

“Fatty acid,” as used herein, refers to any of the members of a large group of monobasic acids, especially those found in animal and vegetable fats and oils. In some embodiments the fatty acid is straight or branched chain alkyl or alkenyl group having 6 to 22 carbons, wherein the carboxylic acid is at one terminus of the carbon chain.

“Glycerides,” as used herein, refers to esters formed between one or more acids and glycerol. In some embodiments, the acids are fatty acids. Medium-chain glycerides are glycerol esters of medium-chain fatty acids containing from 6 to 12 carbon atoms, or, in some embodiments, 6 to 10 carbon atoms. Medium chain fatty acids include: caproic acid (C₆); caprylic acid (C₈), capric acid (C₁₀), and lauric acid (C₁₂). Long chain glycerides are glycerol esters of long chain fatty acids containing from 12 to 22 carbon atoms, or in some embodiments, 12 to 18 carbon atoms.

“Lipid,” as used herein, refers to any of a group of organic compounds, including, but not limited to the fats, oils, waxes, sterols, and triglycerides, that are insoluble in water but soluble in non-polar organic solvents, and are oily to the touch.

As used herein, “microsponge” refers to a porous material capable of adsorbing or absorbing liquids

As used herein, the term “non-miscible liquid” refers to a liquid that does not dissolve in another liquid. Non-miscible liquids are capable of forming emulsions.

“Particulate state,” as used herein, refers to insoluble particles of a given material.

“Phospholipid,” as used herein, refers to phosphorous-containing lipids that are composed mainly of fatty acids, a phosphate group, and a simple organic molecule, e.g. glycerol. Phospholipids may also be referred to as phosphatides.

As used herein, “poorly water-soluble” or “water insoluble” refers to materials, such as an API, that have a solubility in water of less than about 20 mg/mL, less than about 10 mg/mL, less than about 1 mg/mL, less than about 0.1 mg/mL, less than about 0.01 mg/mL, or less than about 0.001 mg/mL at ambient temperature and pressure, and at about pH 7.

Povidone, as used herein, is a polymer of 1-vinyl-2-pyrrolidinone, and having a wide range of average molecular weight. In some embodiments, the povidone has an average molecular weight of from about 2,500 g/mol to about 300,000 g/mol, or greater.

Relative exposure is a percentage value based upon AUC measurements. The percentage is calculated by assigning an AUC value, a value of 100% and expressing the other AUC values as a percentage of the 100% AUC value.

As used herein, “solubilized state,” refers to a solution phase material, such as an API. Such solution phases include solubilization of the API in a solvent, including water, or solubilization of the API in one or more liquid components of an emulsion.

“Sorbitan,” as used herein, refers to dehydrated Sorbitol.

“Starch” refers to a complex carbohydrate consisting of amylase and amylopectin. “Pregelatinized starch” is starch that has been chemically and/or mechanically processed to rupture all or part of the granules in the presence of water and subsequently dried. Some types of pregelatinized starch may be modified to render them compressible and flowable in character.

“Sugar fatty acid,” as used herein, refers to a fatty acid with a sugar moiety attached.

The term “subject,” as used herein, refers to any animal that can experience the beneficial effects of the formulations and methods embodied herein. Preferably, the animal is a mammal, and in particular a human, although it is not intended to be so limited. Examples of other suitable animals include, but are not limited to, rats, mice, monkeys, dogs, cats, cattle, horses, pigs, sheep, and the like.

“Therapeutically effective amount” as used herein with respect to an API dosage shall mean that dosage that provides the specific pharmacological response for which the API is administered in a significant number of subjects in need of such treatment. It is emphasized that “therapeutically effective amount,” administered to a particular subject in a particular instance may not be effective for 100% of patients treated for a specific disease, and will not always be effective in treating the diseases described herein, even though such dosage is deemed a “therapeutically effective amount” by those skilled in the art.

It will be readily understood by those of skill in the art, that some materials identified below as belonging to a category such as an adsorbent carrier, polymeric carriers, phospholipid carriers, pharmaceutically acceptable additives, or other carriers or additives may fall into one or more of those categories, although the material is listed in only one category. For example, magnesium aluminum silicate is both an adsorbent carrier and a synthetic or semi synthetic polymeric carrier. As another example, cellulose may be an adsorbent carrier and a polymeric carrier. Other such materials belonging in more than one category, but listed in only one category, will be readily identified by one of skill in the art.

### B. Multi-Phasic Compositions and Fed/Fasted Variability

Many administered drugs, especially those in oral dosage forms, are susceptible to bioavailability variations due to the presence, or absence of food in the subject's digestive system. Such variability may be evidenced by changes in the AUC, Tₘₐₓ, Cₘₐₓ, or relative exposure when comparing values determined for a subject before and after feeding. The multi-phasic compositions of the present invention may be used to significantly reduce, or in some cases eliminate, such variability for a wide range of drugs.

Multi-phasic compositions are versatile vehicles for a wide variety of active pharmaceutical ingredients, and can be used for the delivery of poorly water-soluble compounds. For example, poorly water-soluble pharmaceuticals tend to be very difficult to deliver to a patient. However, multi-phasic compositions comprising both particulate state API and solubilized state API provide a new route for oral, buccal, vaginal, intranasal, parenteral, or rectal administration for such pharmaceuticals.

Multi-phasic compositions may be described in some embodiments as comprising a drug that is distributed in different phases, or forms, within the same composition, for example as a micro- or nano-particulate form, and/or as a solubilized form (within e.g., an oil, solvent, and/or micelle). Such compositions present the API with significantly enhanced the surface area—principally due to its distribution within multiple phases (e.g., solid nanoparticle, nanoemulsion and/or micelle). In various embodiments, the API is: (i) completely soluble, (ii) completely insoluble, and/or (iii) partially soluble within the vehicle. This phase variation aids in improving bioavailability and in reducing fed/fast variability in oral dosage formulations. In some embodiments, the oral dosage formulation is a solid dosage form, and in other embodiments it is a liquid dosage form.

In one embodiment of the invention, the difference between the AUC, Cₘₐₓ, Tₘₐₓ, or any combination thereof, of a drug administered under fasting conditions, as compared to the same drug (and same drug quantity) administered under fed conditions, preferably administered to a mammal such as a human, is less than about 1000%, less than about 900%, less than about 800%, less than about 700%, less than about 600%, less than about 500%, less than about 400%, less than about 300%, less than about 200%, less than about 100%, less than about 90%, less than about 80%, less than about 70%, less than about 60%, less than about 50%, less than about 40%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%.

In yet another embodiment of the invention, a composition of the invention administered under fed conditions is bioequivalent to the same composition administered under fasting conditions, to a mammal, such as a human. In another embodiment of the invention, “bioequivalency” is defined pursuant to regulatory guidelines. Under United Stated Food and Drug Administration (U.S. FDA) guidelines, two products or methods are bioequivalent if the 90% Confidence Intervals (CI) for AUC and Cₘₐₓ are between 0.80 to 1.25 (Tₘₐₓ measurements are not relevant to bioequivalence for regulatory purposes). The European Medicine's Agency (EMEA) has recently adopted the U.S. FDA guidelines, as previously EMEA guidelines to show bioequivalency required a 90% CI for AUC of between 0.80 to 1.25 and a 90% CI for Cₘₐₓ of between 0.70 to 1.43.

Without being bound to such limitations, the examples provided below illustrate the extent to which the bioavailability variations may be reduced between a fed and a fasted state in rat models. Thus in some embodiments, where the API is fenofibrate, the formulation, when tested in a rat or rat model, may provide a change in the mean AUC between a fed and a fasted state of less than about 90,000 h*ng/ml, less than about 85,000 h*ng/ml, less than about 80,000 h*ng/ml, less than about 75,000 h*ng/ml, less than about 70,000 h*ng/ml, less than about 65,000 h*ng/ml, or less than about 60,000 h*ng/ml.

Relative exposure may also be used to express fed/fasted variability. Thus, in some embodiments, where the active agent is any API described herein, including but not limited to where the active agent is fenofibrate, the formulation may provide a change in the relative exposure between a fed and a fasted state of less than about 1000%, less than about 900%, less than about 800%, less than about 700%, less than about 600%, less than about 500%, less than about 400%, less than about 300%, less than about 200%, less than about 100%, less than about 90%, less than about 80%, less than about 70%, less than about 60%, less than about 50%, less than about 40%, less than about 35%, less than about 30%, less than about 25%, less than about 20%, less than about 15%, less than about 14%, less than about 13%, less than about 12%, less than about 11%, less than about 10%, less than about 9%, less than about 8%, less than about 7%, less than about 6%, less than about 5%, less than about 4%, or less than about 3%.

In other embodiments, the invention provides for formulations wherein: (a) the active pharmaceutical ingredient is any active agent described herein, including but not limited to fenofibrate; and (b) when administered to a mammal, the formulation provides a minimal difference in the mean AUC, mean Cₘₐₓ, and/or mean Tₘₐₓ between a fed and a fasted state.

In yet other embodiments, when tested in a rat or a rat model, a formulation of the invention exhibits a difference in the mean AUC between a fed and a fasted state selected from the group consisting of less than about 90,000 h*ng/ml, less than about 85,000 h*ng/ml, less than about 80,000 h*ng/ml, less than about 75,000 h*ng/ml, less than about 70,000 h*ng/ml, less than about 65,000 h*ng/ml, less than about 60,000 h*ng/ml, less than about 55,000 h*ng/ml, less than about 50,000 h*ng/ml, less than about 45,000 h*ng/ml, less than about 40,000 h*ng/ml, less than about 35,000 h*ng/ml, less than about 30,000 h*ng/ml, less than about 25,000 h*ng/ml, less than about 20,000 h*ng/ml, less than about 15,000 h*ng/ml, and less than about 10,000 h*ng/ml.

The differences in AUC between fed and fasted states may be expressed in a number of ways, including, but not limited to a percentage difference between any two determined AUC values, or the difference between relative exposure values. Thus, in some embodiments, upon administration to a mammal, a percent difference between a mean AUC, mean Cₘₐₓ, and/or mean Tₘₐₓ determined at a fasted state and a mean AUC, mean Cₘₐₓ, and/or mean Tₘₐₓ determined at a fed state is less than about 1000%, less than about 900%, less than about 800%, less than about 700%, less than about 600%, less than about 500%, less than about 400%, less than about 300%, less than about 200%, less than about 100%, less than about 90%, less than about 80%, less than about 70%, less than about 60%, less than about 50%, less than about 40%, less than about 30%, less than about 20%, less than about 10%. In other embodiments, the percent difference is selected from the group consisting of less than about 9%, less than about 8%, less than about 7%, less than about 6%, less than about 5%, less than about 4%, less than about 3%, less than about 2%, less than about 1%, and less than about 0.05%.

In other embodiments of the invention, the invention provides for formulations wherein: (a) the active pharmaceutical ingredient can be, but is not limited to, fenofibrate; and (b) upon administration to a mammal, the formulation exhibits a difference in the relative exposure between a fed and a fasted state of less than about 1000%. In yet other embodiments, the formulation exhibits a difference in the relative exposure between a fed and a fasted state selected from the group consisting of less than about 900%, less than about 800%, less than about 700%, less than about 600%, less than about 500%, less than about 400%, less than about 300%, less than about 200%, less than about 100%, less than about 90%, less than about 80%, less than about 70%, less than about 60%, less than about 50%, less than about 45%, less than about 40%, less than about 35%, less than about 30%, less than about 25%, less than about 20%, less than about 15%, less than about 10%, less than about 5%, less than about 4%, and less than about 3%.

### C. Multi-Phasic Compositions in Solid and Liquid Forms

In one aspect, pharmaceutical formulations are provided comprising a multi-phasic pharmaceutical composition in an oral dosage form comprising (1) an active pharmaceutical ingredient, wherein the active pharmaceutical ingredient is in a particulate state, a solubilized state, or in both a particulate state and in a solubilized state; (2) a solvent; (3) a non-miscible liquid; (4) a stabilizer; and (5) water. In such pharmaceutical formulations, the multi-phasic pharmaceutical composition is preferably present at about 1 to about 90 wt %.

The oral dosage forms of the pharmaceutical formulations embodied herein, may be in solid or liquid dosage forms. Such solid or liquid forms may be formulated into suitable dosage forms known to those of skill in the art such as a capsule, emulsion, tablet, and the like. In some embodiments, the multi-phasic pharmaceutical composition is present in the pharmaceutical formulation at about 0.1 wt % to about 90 wt %. In some embodiments, the API is present in the pharmaceutical formulation at about 0.1 to about 70 wt %.

In multi-phasic pharmaceutical compositions, when the API is present in both a particulate state and in a solubilized state, the amount of an API in a particulate state and the amount of an API in a solubilized state may vary. In some embodiments, the amount of API in the particulate state ranges from about 5 wt % to about 95 wt %, from about 10 wt % to about 90 wt %, from about 15 wt % to about 85 wt %, from about 20 wt % to about 80 wt %, from about 25 wt % to about 78 wt %, from about 30 wt % to about 75 wt %, from about 35 wt % to about 73 wt %, from about 40 wt % to about 70 wt %, from about 45 wt % to about 70 wt %, from about 50 wt % to about 70 wt %, from about 60 wt % to about 70 wt %, and/or from about 65 wt % to about 70 wt %. In some embodiments, the amount of API in the solubilized state ranges from about 0.5 wt % to about 80 wt %, from about 1.0 wt % to about 75 wt %, from about 5 wt % to about 70 wt %, from about 10 wt % to about 65 wt %, from about 15 wt % to about 60 wt %, from about 20 to about 55 wt %, from about 25 wt % to about 50 wt %, from about 25 wt % to about 45 wt %, from about 25 wt % to about 40 wt %, from about 28 wt % to about 35 wt %, and/or from about 28 wt % to about 33 wt %. The amount of API in a particulate state and the amount of API in a solubilized state for a multi-phasic composition may also be expressed as a weight ratio of the amount of API in a particulate state to the amount of API in the solubilized state. For example, such a ratio may range from about 95:5 to about 5:95. In some embodiments, the ratio is about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, about 10:90, or about 5:95.

In instances where the oral dosage form is a solid dosage form, the pharmaceutical formulations embodied herein comprise a multi-phasic pharmaceutical composition and an adsorbent carrier. Without being bound by theory, adsorbent carriers adsorb the non-miscible liquid (in some embodiments, an oil) that is present in the multi-phasic pharmaceutical composition to aid in the formation of a solid dosage form pharmaceutical formulation. Suitable adsorbent carriers for use in the embodied pharmaceutical formulations include porous materials, clays, silicates, cellulose-based polymers, microsponges, other synthetic polymers, or mixtures of any two or more thereof. Exemplary clays include attapulgite, bentonite, kaolin, perlite, talc, vermiculites, zeolites, or a mixture of any two or more thereof. Exemplary silicates include aluminum silicate, magnesium aluminum silicate, hydrous calcium silicate, colloidal silicon dioxide, magnesium aluminometasilicate, and mixtures of any two or more thereof. Exemplary cellulose-based polymers include carboxymethyl cellulose calcium, carboxymethyl cellulose sodium, cellulose, cellulose acetate, cellulose acetate phthalate, ethyl cellulose, hydroxyethyl cellulose, hydroxypropyl cellulose, hydroxypropyl methylcellulose, hydroxypropyl methylcellulose phthalate, methylcellulose, microcrystalline cellulose, powdered cellulose, or a mixture of any two or more thereof. Other synthetic polymers suitable for use as adsorbent carriers include cross-linked acrylic polymers, polypropylene, polyurethane foams, or mixtures of any two or more thereof.

Other adsorbent carriers that may be used in the embodied solid dosage forms include, but are not limited to, calcium carbonate, calcium phosphate dibasic anhydrous, calcium phosphate dibasic dehydrate, calcium phosphate tribasic, calcium sulfate, lactose, magnesium carbonate, magnesium oxide, mannitol, silicon dioxide, sodium starch glycolate, sodium chloride, sorbitol, starch, sucrose, or a mixture of any two or more thereof.

Other carriers and additives may also be included in the embodied solid dosage forms. Such other carriers and additives may be used to give binding, coloring, compressing, filling, flavoring, lubricating, and/or preserving properties to the pharmaceutical formulations or they may be used for other purposes known to those of skill in the art. For example, other carriers and additives may include, but are not limited to polymeric carriers, phospholipid carriers, lubricants, antioxidants, coloring agents, flavoring agents, preservatives, sweeteners, volatile oils, and/or a mixture of any two or more thereof.

Exemplary polymeric carriers that may be used in the embodied pharmaceutical formulations include, but are not limited to, carbomers, croscarmellose sodium, crospovidone, cyclodextrins, β-cyclodextrins, Docusate sodium, hydroxypropyl-β-cyclodextrins, γ-cyclodextrins, polyanionic-β-cyclodextrins, sulfobutylether-7-β-cyclodextrin, methacrylic acid copolymers, poloxamer, polydextrose, polyethylene oxide, polymethacrylate polymers, poly(methacrylic acid-methyl methacrylate), poly(methacrylic acid-ethyl acrylate), ammonio methacrylate copolymer, poly(ethyl acrylate-methylmethacrylate-trimethylammonioethyl methacrylate chloride), poly(ethyl acrylate-methyl methacrylate), polysaccharides, polyvinyl alcohol with an average molecular weight of from about 20,000 to about 200,000 g/mol, polyvinylpyrrolidine/vinyl acetate, povidone with an average molecular weight of from about 2,500 to about 300,000 g/mol, poloxamer, sodium starch glycolate, or a mixture of any two or more thereof. Exemplary polysaccharides include, but are not limited to, acacia, alginic acid, carrageenan, ceratonia, chitosan, compressible sugar, confectioner's sugar, dextrates, dextrin, dextrose, fructose, fumaric acid, gelatin, glucose, liquid, glyceryl behenate, guar gum, lactitol, lactose, maltodextrin, maltose, mannitol, polydextrose, polymethacrylates, pregelatinized starch, sodium alginate, sorbitol, starch, pregelatinized starch, sterilizable maize, sucrose, sugar spheres, tragacanth, trehalose, xylitol, or a mixture of any two or more thereof.

Some of the polymeric carriers may also be variously known in the art as disintegrants, compression aids, or binders. For example, disintegrants may include, but are not limited to, cellulose-based polymers; polysaccharides; other materials such as croscarmellose sodium, crospovidone, docusate sodium, magnesium aluminum silicate, colloidal silicon dioxide, calcium phosphate tribasic, povidone; or a mixture of any two or more thereof, as well as other materials and mixtures known to those of skill in the art to be useful as disintegrants. Compression aids may include, but are not limited to, polysaccharides and cellulose-based polymers and also non-polymeric materials such as inorganic salts, including but not limited to, calcium carbonate, calcium phosphate, calcium sulfate, magnesium carbonate, magnesium oxide, sodium chloride. Binders may also include materials such as polysaccharides and other synthetic or semi-synthetic polymers.

Exemplary phospholipid carriers that may be used in the embodied pharmaceutical formulations include, but are not limited to, diphosphatidylglycerol, glycolipids, phosphatidic acid, phosphatidylcholine, phosphatidylethanolamine, phosphatidylglycerol, phosphatidylinositol, phosphatidylserine, sphingomyelin, or a mixture of any two or more thereof. Exemplary lubricants include magnesium stearate, talc, stearic acid, calcium stearate, zinc stearate, glyceryl palmitostearate, glyceryl behenate, light mineral oil, micronized poloxamers, polyethylene glycol, l-leucine, vegetable oil.

The liquid and/or solid dosage forms embodied herein may also include pharmaceutically acceptable additives such as, but not limited to, an antioxidant, a coloring agent, a flavoring agent, a preservative, a sweetener, a volatile oil, or a mixture of any two or more thereof. Exemplary antioxidants include, but are not limited to, ascorbic acid, ascorbyl palmitate, butylated hydroxyanisole, butylated hydroxytoluene, ethylenediaminetetraacetic acid, salts of ethylenediaminetetraacetic acid, propyl gallate, sodium metabisulfite, vitamin E, esters of vitamin E, or a mixture of any two or more thereof. Exemplary preservatives include, but are not limited to, butylparaben, calcium sorbate, ethylparaben, methylparaben, monothioglycerol, potassium sorbate, propylparaben, sodium benzoate, sodium sorbate, sorbic acid, or a mixture of any two or more thereof. Exemplary sweeteners include, but are not limited to, aspartame, glycyrrhizin salts, monoammonium glycyrrhizinate, saccharin, saccharin calcium, saccharin sodium, sugar, sucralose, or a mixture of any two or more thereof. Exemplary flavoring agents include, but are not limited to, anise, banana, cherry, chocolate, citric acid, lemon, menthol, orange, peppermint, pineapple, rum, sodium citrate, strawberry, vanillin, ethyl vanillin, or a mixture of any two or more thereof. Exemplary coloring agents include, but are not limited to, FD&C blue #1, FD&C blue #2, FD&C green #3, FD&C red #3, FD&C red #4, FD&C yellow #5, FD&C yellow #6, D&C blue #4, D&C green #5, D&C green #6, D&C orange #4, D&C orange #5, iron oxides, or a mixture of any two or more thereof. Exemplary volatile oils include, but are not limited to, balm oil, bay oil, bergamot oil, cedarwood oil, cherry oil, cinnamon oil, clove oil, origanum oil, peppermint oil, or a mixture of any two or more thereof.

The use of solid dosage forms such as capsules, tablets, lozenges, and/or cachets is well known in the art for the oral, buccal, or rectal administration of a pharmaceutical agent to a subject. The pharmaceutical formulations embodied herein, may be used in the preparation of such capsules, tablets, lozenges, and/or cachets. Capsules may be hard or soft, and may be made of a variety of materials known to those of skill in the art, including, but not limited to, cellulose materials, gelatin, carrageenan, agar, and pectin. When such solid dosage forms are placed in aqueous media, the formulations disintegrate to release the active pharmaceutical ingredient.

The use of liquid dosage forms such as solutions, emulsions, suspensions, syrups, elixirs, capsules, and the like is well known in the art for the oral administration of a pharmaceutical agent to a subject. The pharmaceutical formulations embodied herein, may be used in the preparation of such solutions, emulsions, capsules, and the like. Capsules may be hard or soft, and may be made of a variety of materials known to those of skill in the art, including, but not limited to, cellulose materials, gelatin, carrageenan, agar, and pectin.

Active pharmaceutical ingredients useful in the embodied multi-phasic pharmaceutical compositions include any suitable API for multi-phasic compositions. For example, suitable APIs may include, but are not limited to agents used in the treatment of AIDS, agents used in treatment of heart disorders, analgesics, anesthetics, anorexiants, anthelmintics, anti-allergic agents, anti-anginal agents, antiarrhythmic agents, anticholinergics, anticoagulants, antidepressants, antidiabetic agents, antidiuretic agents, anti-emetic agents, antiepileptics, anti-fungals, antihistamines, anti-hypertensive agents, anti-inflammatory agents, anti-migraine agents, antimuscarinic agents, antimycobacterial agents, antineoplastic agents including, antiparkinsonian agents, antithyroid agents, antiviral agents, astringents, blocking agents, blood products, blood substitutes, cardiac inotropic agents, cardiovascular agents, central nervous system agents, chelating agents, chemotherapy agents, colony stimulating factors, corticosteroids, cough suppressants, dermatological agents, diuretics, dopaminergics, elastase inhibitors, endocrine agents, ergot alkaloids, expectorants, gastrointestinal agents, genitounnary agents, growth hormone releasing hormone, growth hormones, hematological agents, hematopoietic agents, hemostatics, hormones, immunologic agents, immunosuppressants, interleukins, interleukin analogues, lipid regulating agents, luteinizing hormone releasing hormone, muscle relaxants, narcotic antagonists, nutrients, nutritional agents, oncology therapies, organic nitrates, parasympathomimetics, prostaglandins antibiotics, renal agents, respiratory agents, sedatives, sex hormones, stimulants, sympathomimetics, systemic anti-infectives, tacrolimus, thrombolytic agents, thyroid agents, treatments for attention deficit disorder, uterine-active agents, vaccines, vasodilators, xanthines, cholesterol lowering agents, biotechnology products, including but not limited to proteins, peptides, and antibodies, or mixtures of any two or more thereof. Specific examples of API will be readily recognized by one of skill in the art, and may include, but are not limited to, raloxifene, an antiviral compound such as acyclovir, a compound useful in the relief of symptoms associated with perennial and seasonal allergic rhinitis; vasomotor rhinitis; allergic conjunctivitis; mild, uncomplicated urticaria and angioedema; or the amelioration of allergic reactions to blood or plasma; or dermatographism or as adjunctive therapy in anaphylactic reactions. Examples of such compounds include, but are not limited to, loratidine, desloratidine, and cetirizine. Mixtures of any two or more of the above APIs identified by name or category are also embodied herein.

In some embodiments, the active pharmaceutical ingredient is naltrexone, alendronic acid, nicotine, testosterone, progesterone, estradiol, fenofibrate, danazol, naproxen, sildenafil, griseofulvin, mycophenolate mofetil, an immunosuppressant such as cyclosporine or sirolimus, or a mixture of any two or more thereof.

Solvents useful in the embodied pharmaceutical formulations include, but are not limited to, an alcohol, N-methylpyrrolidinone, methoxypolyethylene glycol, polyethylene glycol, polyethylene oxide, ethoxy diglycol, triacetin, dimethyl sulfoxide, propylene glycol, isopropyl myristate, mono-, di- or tri-glycerides, or a mixture of any two or more thereof. Exemplary alcohols include benzyl alcohol, ethyl alcohol, or a mixture of any two or more thereof. Exemplary polyethylene glycols have an average molecular weight of about 1000 g/mol or greater, and the methoxypolyethylene glycol has an average molecular weight of about 1000 g/mol or greater. In other embodiments, the polyethylene glycol has an average molecular weight of from about 1000 g/mol to about 20,000 g/mol, and the methoxypolyethylene glycol has an average molecular weight of from about 1000 g/mol to about 20,000 g/mol.

Non-miscible liquids for use in the embodied pharmaceutical formulations include, but are not limited to, fatty acids, medium chain glycerides, long chain glycerides, ethyl esters of a fatty acid, propylene glycol fatty acid esters, sorbitan fatty acid esters, polyglyceryl fatty acid esters, glyceryl mono-, di-, or tri-caprylic acid esters; glyceryl mono-, di-, or tri-capric acid esters; or a mixture of any two or more thereof. Non-miscible liquids also include vegetable oils, nut oils, fish oils, lard oil, mineral oils, squalane, tricaprylin (1,2,3-trioctanoyl glycerol), and mixtures of any two or more thereof. For example, almond oil (sweet), apricot seed oil, borage oil, canola oil, coconut oil, corn oil, cotton seed oil, fish oil, jojoba bean oil, lard oil, linseed oil (boiled), macadamia nut oil, medium chain triglycerides, mineral oil, olive oil, origanum oil, peanut oil, safflower oil, sesame oil, soybean oil, sunflower seed oil, wheat germ oil, mineral oil (light), DL-α-tocopherol, ethyl oleate, ethyl linoleate, glyceryl behenate, glyceryl monooleate, glyceryl monostearate, glyceryl palmitostearate, linoleic acid, linolenic acid, oleic acid, palmitostearic acid, peppermint oil, polyglyceryl oleate, propylene glycol monolaureate, propylene glycol dilaureate, sorbitan monolaurate, sorbitan monooleate, sorbitan monopalmitate, sorbitan monostearate, sorbitan trioleate, stearic acid, tetraglyceryl monooleate, or a mixture of any two or more thereof are all examples of non-miscible liquids for use in the embodied pharmaceutical formulations.

Stabilizers useful in the embodied pharmaceutical formulations include, but are not limited to, non-phospholipid surfactants, non-phenol polyethylene glycol ethers, sorbitan esters, polyethylene glycol esters, block polymers, acrylic polymers, ethoxylated fatty acids, ethoxylated alcohols, ethoxylated fatty acid esters, monoglycerides, silicon-based surfactants, polysorbates, tergitols, sugar fatty acid ester; a sucrose mono-, di-, or tri-fatty acid ester; a polyoxyethylene castor oil compound; a polyoxyethylene sorbitan fatty acid ester; a polyoxyethylene mono- or di-fatty acid ester; a polyoxyethylene alkyl ether; a glyceryl mono-, di-, or tri-fatty acid ester; a mixtures of polyoxyethylene mono- or di-ester of a C₈-C₂₂ fatty acid; a glyceryl mono-, di-, or tri-ester of a C₈-C₂₂ fatty acid, or a mixture of any two or more thereof. For example, the stabilizer may be ARLACEL™, BRIJ™, Cremophore RH-40, glycerin monostearate, PEMULEN™, Pluronics™, polyethylene glycol stearate, polyoxyl 35 castor oil, polyoxyl 40 hydrogenated castor oil, polyoxyl 60 hydrogenated castor oil, polysorbate 20, polysorbate 40, polysorbate 60, polysorbate 80, polyoxyl 40 stearate, polyoxyl 40 oleate, polyoxyl 20 cetostearyl ether, polyoxyl 10 oleyl ether, sodium dioctyl sulfosuccinate, sodium lauryl sulfate, SPAN™, TERGITOL™ NP-40, TERGITOL™ NP-70, DL-α-tocopheryl polyethylene glycol succinate, TWEEN™ 20, TWEEN™ 60, TWEEN™ 80, or a mixture of any two or more thereof.

Methods of preparing the pharmaceutical formulations are also provided. Such methods comprise mixing an active pharmaceutical ingredient, a solvent, a stabilizer, and a non-miscible liquid to form a first mixture; and emulsifying the first mixture with water to form a multi-phasic pharmaceutical composition that is then formulated as an oral dosage form. When the oral dosage form is a solid dosage form, the method further comprises mixing the emulsified first mixture with an adsorbent carrier. Such methods may further comprise pressing the solid dosage form into a capsule or tablet, or filling a capsule with a liquid dosage form. In such embodied methods, the API may be present at about 0.1 to about 70 wt % of the capsule or tablet.

In some embodied methods and in the embodied pharmaceutical formulations, the multi-phasic composition comprises globules of the non-miscible liquid and the globules have a diameter of less than about 10 μm. For example, the globules may have a diameter of less than about 9 microns, less than about 8 microns, less than about 7 microns, less than about 6 microns, less than about 5 microns, less than about 4 microns, less than about 3 microns, less than about 2 microns, less than about 1000 nm, less than about 900 nm, less than about 800 nm, less than about 700 nm, less than about 600 nm, less than about 500 nm, less than about 400 nm, less than about 300 nm, less than about 290 nm, less than about 280 nm, less than about 270 nm, less than about 260 nm, less than about 250 nm, less than about 240 nm, less than about 230 nm, less than about 220 nm, less than about 210 nm, less than about 200 nm, less than about 190 nm, less than about 180 nm, less than about 170 nm, less than about 160 nm, less than about 150 nm, less than about 140 nm, less than about 130 nm, less than about 120 nm, less than about 110 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm.

In some embodied methods and pharmaceutical formulations, the multi-phasic composition comprises at least a portion of the API in particulate form. In some embodiments, the average diameter of the particles of the particulate form is from about 1 nm to about 10 microns. In some embodiments, the average diameter of the particles of the particulate form is less than about 10 microns. For example, the average diameter of the particles may be less than about 9 microns, less than about 8 microns, less than about 7 microns, less than about 6 microns, less than about 5 microns, less than about 4 microns, less than about 3 microns, less than about 2 microns, or about 1 micron or greater. In other embodiments, the average diameter of the particles is less than about 1 micron, such as from about 1 nm to about 1 micron. For example, the diameter of the API particles may be less than about 900 nm, less than about 800 nm, less than about 700 nm, less than about 600 nm, less than about 500 nm, less than about 400 nm, less than about 300 nm, less than about 290 nm, less than about 280 nm, less than about 270 nm, less than about 260 nm, less than about 250 nm, less than about 240 nm, less than about 230 nm, less than about 220 nm, less than about 210 nm, less than about 200 nm, less than about 190 nm, less than about 180 nm, less than about 170 nm, less than about 160 nm, less than about 150 nm, less than about 140 nm, less than about 130 nm, less than about 120 nm, less than about 110 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm.

One of ordinary skill will appreciate that effective amounts of an API can be determined empirically and can be employed in pure form or, where such forms exist, in pharmaceutically acceptable salt, ester, or prodrug form. Actual dosage levels of an API in the nanoparticulate compositions of the invention may be varied to obtain an amount of the API that is effective to obtain a desired therapeutic response for a particular composition and method of administration. The selected dosage level therefore depends upon the desired therapeutic effect, the route of administration, the potency of the administered API, the desired duration of treatment, and other factors.

Dosage unit compositions may comprise such amounts or submultiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient; the time of administration, route of administration, and rate of excretion of the agent; the duration of the treatment; drugs used in combination or coincidental with the specific agent; and like factors well known in the medical arts.

One skilled in the art will readily realize that all ranges and ratios discussed can and do necessarily also describe all subranges and subratios therein for all purposes and that all such subranges and subratios also form part and parcel of this invention. Any listed range or ratio can be easily recognized as sufficiently describing and enabling the same range or ratio being broken down into at least equal halves, thirds, quarters, fifths, tenths, etc. As a non-limiting example, each range or ratio discussed herein can be readily broken down into a lower third, middle third and upper third, etc.

All publications, patent applications, issued patents, and other documents, if any, referred to in this specification are herein incorporated by reference as if each individual publication, patent application, issued patent, or other document was specifically and individually indicated to be incorporated by reference in its entirety, for all purposes. Definitions that are contained in text incorporated by reference are excluded to the extent that they contradict definitions in this disclosure.

The present invention, thus generally described, will be understood more readily by reference to the following examples, which are provided by way of illustration and are not intended to be limiting of the present invention.

### EXAMPLES

### Formulation I: Control

Non-micronized fenofibrate powder was suspended in hydroxypropyl methylcellulose (HPMC, grade E4M) to give a 0.5 wt % suspension (48 mg of fenofibrate per gram of suspension). The suspension was mixed very well to ensure a uniform suspension free from lumps and/or aggregates.

### Formulation II: Standard

A TriCor tablet (48 mg fenofibrate per tablet, available from Abbott Pharma) was powered using a mortar and pestle to until an aggregate-free mass was obtained. The mass was then suspended in one milliliter of purified water to obtain a uniform suspension.

### Formulation III: Test I

Fenofibrate (4.8 gm) was mixed with ethanol (8.8 gm), polysorbate 80 (9.4 gm), and soybean oil (50.2 gm). Water (26.8 gm) was added and the entire mixture subjected to emulsification using a paddle-type stirrer. The resultant emulsion was then subjected to high-pressure homogenization (APV-1000) at 10,000 psi for three cycles.

### Formulation IV: Test II

Fenofibrate (4.8 g) was mixed with ethanol (15 g) and medium chain triglycerides (40.0 g, Crodamol GTCC). The mixture was warmed (40° C.) to dissolve the fenofibrate with gentle mixing. Separately, poloxamer 188 (7.0 g) was dissolved in water (33.2 g) to form a solution which was then added to the fenofibrate solution. The resulting mixture was subjected to emulsification using a paddle-type stirrer. The emulsion was further subjected to high-pressure homogenization (APV-1000) at 10,000 psi for three cycles.

preclinical study

The investigation was carried out in rats by administering Formulations I-IV using an oral gavage at a dose of 90 mg fenofibrate per kilogram body weight of animal, and then observing the blood concentrations of fenofibric acid as a function of time. Fenofibric acid is the active, primary metabolite resulting from the administration of fenofibrate to a subject.

### Phase I: Demonstration of Improved Bioavailability Compared to Control

In the first phase, the control formulation, the standard formulation, and the Test I formulations were compared under fasted conditions. Five rats per group were used in this initial study. Each rat was given a single dose of 90 mg/kg of fenofibrate. The area under the plasma concentration-time curve (AUC) over a 24 period (correlating to the amount of drug absorbed or bioavailability), Cₘₐₓ (maximum concentration of the drug in the blood), and Tₘₐₓ (time to reach Cₘₐₓ) were measured for each of the three groups, and the data is presented in Table 1 below.

TABLE 1
Fenofibrate Bioavailability Data in Fasted Rats

| | AUC_{24 h} | | Cₘₐₓ | | Tₘₐₓ | |
|---|---|---|---|---|---|---|
| Group | (hr * ng/mL) | | (ng/mL) | | (hr) | |
| (Formulation) | Mean | SD | Mean | SD | Mean | SD |
| Control | 216,542.1 | 125,241.2 | 31,080.0 | 7851.9 | 4.4 | 2.2 |
| Standard | 1,480,971.8*^{#} | 333,521.8 | 180,600.0 | 34,121.8 | 3.6 | 2.6 |
| Test I | 912,679.9* | 161,665.7 | 132,500.0 | 19,710.4 | 4.0 | 0.0 |

*Statistically significant increase compared to Control (p < 0.05, two-tailed t-test)
^{#}Statistically significant increase compared to Test I (p < 0.05, two-tailed t-test)

The dose and mean AUC were then used to compute the relative exposure (%) for each group. “Relative exposure” represents the extent of overall bioavailability of an API in a subject. The relative exposure projects how test and control formulations perform with respect to the formulation which gave best results. In this case the formulation with the best results was the Standard formulation (100%) to which the other formulations are normalized. This data is presented in Table 2.

TABLE 2
Fenofibrate Relative Exposure in Fasted Rats.

| Group | Dose | Mean AUC | Relative |
|---|---|---|---|
| (Formulation) | (mg/kg) | (h * ng/mL) | Exposure % |
| Standard | 90 | 1480971.8 | 100.0 |
| Test I | 90 | 912679.9 | 61.6 |
| Control | 90 | 216542.1 | 14.6 |

Tables 1 and 2 show that both the Standard and Test I formulations present an improvement in oral bioavailability of Fenofibrate when compared to the Control formulation. The Standard formulation offered statistically significant higher bioavailability as compared to the Test I formulation.

### Phase II: Elimination of Fed/Fast Variability

In the second phase, the Standard and the Test II Formulations were compared under fed and fasted conditions. Rats were divided into four groups of five rats each: (i) Standard—fasted, (ii) Standard—fed, (iii) Test II—fasted, and (iv) Test II—fed. Each of the formulations, in an amount equivalent to a fenofibrate dose of 90 mg/Kg, was administered as a single oral dose and the resulting blood pharmacokinetics were evaluated (Table 3).

TABLE 3
Fenofibrate Bioavailability in Fed and Fasted Rats

| | AUC_{24 h} | | Cₘₐₓ | | Tₘₐₓ | |
|---|---|---|---|---|---|---|
| | (h * ng/mL) | | (ng/mL) | | (h) | |
| Group | Mean | SD | Mean | SD | Mean | SD |
| Standard - Fasted | 1245585 | 487453 | 146000 | 49432 | 3.6 | 0.9 |
| Standard - Fed | 1345108 | 311789 | 137200 | 26186 | 3.6 | 0.9 |
| Test II - Fasted | 1862671 | 480725 | 207000 | 28080 | 3.2 | 1.1 |
| Test II - Fed | 1919344 | 274560 | 189400 | 31501 | 3.2 | 1.1 |

As Table 3 illustrates, under both fed and fasted conditions, the difference between the AUC for each of the two formulations was statistically insignificant. In other words, for the Standard formulation there was no variation in bioavailability of fenofibrate in rats between the fed and fasted states. The same was true for the Test II formulation. As above, this data may be presented in terms of relative exposure, as shown in Table 4.

TABLE 4
Fenofibrate Relative Exposure in Fed and Fasted Rats

| | | | Relative Exposure |
|---|---|---|---|
| | Dose | Mean AUC | % All Groups |
| Group | (mg/kg) | (h * ng/mL) | Compared |
| Standard - Fasted | 90 | 1245585 | 64.9 |
| Standard - Fed | 90 | 1345108 | 70.1 |
| Test II - Fasted | 90 | 1862671 | 97.0 |
| Test II - Fed | 90 | 1919344 | 100.0 |

Table 4 clearly illustrates that Test II formulations have a higher relative exposure as compared to the Standard formulations. Expressed as AUC, the higher exposure from the Test II formulation offers a statistically significant improvement as compared to the Standard formulation. Therefore, it may be concluded that the Test II formulation has significant improvements over the currently marketed preparations for Fenofibrate in terms of improving oral bioavailability and reducing fed/fast variability.

We claim:

1. A pharmaceutical formulation comprising a multi-phasic pharmaceutical composition in an oral dosage form, wherein:
(a) the composition exhibits a reduced variability in the mean AUC, mean Cmax, and/or mean Tmax following administration of the composition to a mammal under fed conditions as compared to fasting conditions, as compared to a prior art pharmaceutical composition of the same active pharmaceutical ingredient at the same dosage, comprising:
(b) the composition comprises:
(i) an active pharmaceutical ingredient, wherein the active pharmaceutical ingredient is in a particulate state, a solubilized state, or in both a particulate state and in a solubilized state;
(ii) a solvent;
(iii) a non-miscible liquid;
(iv) a stabilizer; and
(v) water.

2. The pharmaceutical formulation of claim 1, wherein upon administration to a mammal, the formulation exhibits an absorption profile under fed conditions which is similar to, or bioequivalent to, the absorption profile of the same composition administered under fasting conditions.

3. The pharmaceutical formulation of claim 2, wherein the mammal is a human.

4. The pharmaceutical formulation of claim 1, wherein upon administration of the composition to a rat or a rat model, the difference between a mean AUC determined at a fed state and a mean AUC determined at a fasted state is less than about 90,000 h*ng/ml.

5. The pharmaceutical formulation of claim 4, wherein the difference is selected from the group consisting of less than about 85,000 h*ng/ml, less than about 80,000 h*ng/ml, less than about 75,000 h*ng/ml, less than about 70,000 h*ng/ml, less than about 65,000 h*ng/ml, less than about 60,000 h*ng/ml, less than about 55,000 h*ng/ml, less than about 50,000 h*ng/ml, less than about 45,000 h*ng/ml, less than about 40,000 h*ng/ml, less than about 35,000 h*ng/ml, less than about 30,000 h*ng/ml, less than about 25,000 h*ng/ml, less than about 20,000 h*ng/ml, less than about 15,000 h*ng/ml, and less than about 10,000 h*ng/ml.

6. The pharmaceutical formulation of claim 1, wherein upon administration to a mammal, a percent difference between a mean AUC, mean Cₘₐₓ, and/or mean Tₘₐₓ determined at a fasted state and a mean AUC determined at a fed state is less than about 1000%.

7. The pharmaceutical formulation of claim 6, wherein the percent difference is selected from the group consisting of less than about 900%, less than about 800%, less than about 700%, less than about 600%, less than about 500%, less than about 400%, less than about 300%, less than about 200%, less than about 100%, less than about 90%, less than about 80%, less than about 70%, less than about 60%, less than about 50%, less than about 40%, less than about 30%, less than about 20%, less than about 10%, less than about 9%, less than about 8%, less than about 7%, less than about 6%, less than about 5%, less than about 4%, less than about 3%, less than about 2%, less than about 1%, and less than about 0.5%.

8. The pharmaceutical formulation of claim 1, wherein upon administration to a mammal, the formulation exhibits a difference in the relative exposure of the active pharmaceutical ingredient between a fed and a fasted state of less than about 1000%.

9. The pharmaceutical formulation of claim 8, wherein the difference in the relative exposure between a fed and a fasted state is selected from the group consisting of less than about 900%, less than about 800%, less than about 700%, less than about 600%, less than about 500%, less than about 400%, less than about 300%, less than about 200%, less than about 100%, less than about 90%, less than about 80%, less than about 70%, less than about 60%, less than about 50%, less than about 45%, less than about 40%, less than about 35%, less than about 30%, less than about 25%, less than about 20%, less than about 15%, less than about 10%, less than about 5%, less than about 4%, and less than about 3%.

10. The pharmaceutical formulation of claim 1, further comprising one or more compounds selected from the group consisting of adsorbent carriers, viscosity modifiers, and coloring and flavoring agents, wherein the oral dosage form is a solid or liquid oral dosage form.

11. The pharmaceutical formulation of claim 10, wherein the multi-phasic pharmaceutical composition is present at about 0.1 to about 90 wt %.

12. The pharmaceutical formulation of claim 10, wherein the adsorbent carrier is a clay, a silicate, a cellulose-based polymer, a microsponge, other synthetic polymers, or a mixture of any two or more thereof.

13. The pharmaceutical formulation of claim 10, wherein the absorbent carrier is attapulgite, bentonite, kaolin, perlite, talc, vermiculites, zeolites, aluminum silicate, magnesium aluminum silicate, hydrous calcium silicate, colloidal silicon dioxide, magnesium aluminometasilicate, carboxymethyl cellulose calcium, carboxymethyl cellulose sodium, cellulose, cellulose acetate, cellulose acetate phthalate, ethyl cellulose, hydroxyethyl cellulose, hydroxypropyl cellulose, hydroxypropyl methylcellulose, hydroxypropyl methylcellulose phthalate, methylcellulose, microcrystalline cellulose, powdered cellulose, a cross-linked acrylic polymer, a polypropylene, a polyurethane foam, calcium carbonate, calcium phosphate dibasic anhydrous, calcium phosphate dibasic dehydrate, calcium phosphate tribasic, calcium sulfate, lactose, magnesium carbonate, magnesium oxide, mannitol, silicon dioxide, sodium starch glycolate, sodium chloride, sorbitol, starch, sucrose, or a mixture of any two or more thereof.

14. The pharmaceutical formulation of claim 10, further comprising a lubricant, a disintegrant, or a mixture thereof.

15. The pharmaceutical formulation of claim 14, wherein the lubricant is magnesium stearate, talc, stearic acid, calcium stearate, zinc stearate, glyceryl palmitostearate, glyceryl behenate, light mineral oil, micronized poloxamers, polyethylene glycol, l-leucine, vegetable oil, or a mixture of any two or more thereof.

16. The pharmaceutical formulation of claim 10, wherein the solid dosage form is a capsule or tablet.

17. The pharmaceutical formulation of claim 10, wherein upon deposition in an aqueous medium, the pharmaceutical formulation disintegrates to release the active pharmaceutical ingredient.

18. The pharmaceutical formulation of claim 1, wherein the oral dosage form is a liquid dosage form.

19. The pharmaceutical formulation of claim 18, wherein the liquid dosage form is a solution, an emulsion, a suspension, a syrup, or an elixir.

20. The pharmaceutical formulation of claim 1, wherein the active pharmaceutical ingredient is selected from agents used in the treatment of AIDS, agents used in treatment of heart disorders, analgesics, anesthetics, anorexiants, anthelmintics, anti-allergic agents, anti-anginal agents, antiarrhythmic agents, anticholinergics, anticoagulants, antidepressants, antidiabetic agents, antidiuretic agents, anti-emetic agents, antiepileptics, anti-fungals, antihistamines, anti-hypertensive agents, anti-inflammatory agents, anti-migraine agents, antimuscarinic agents, antimycobacterial agents, antineoplastic agents including, antiparkinsonian agents, antithyroid agents, antiviral agents, astringents, blocking agents, blood products, blood substitutes, cardiac inotropic agents, cardiovascular agents, central nervous system agents, chelating agents, chemotherapy agents, colony stimulating factors, corticosteroids, cough suppressants, dermatological agents, diuretics, dopaminergics, elastase inhibitors, endocrine agents, ergot alkaloids, expectorants, gastrointestinal agents, genitounnary agents, growth hormone releasing hormone, growth hormones, hematological agents, hematopoietic agents, hemostatics, hormones, immunologic agents, immunosuppressants, interleukins, interleukin analogues, lipid regulating agents, luteinizing hormone releasing hormone, muscle relaxants, narcotic antagonists, nutrients, nutritional agents, oncology therapies, organic nitrates, parasympathomimetics, prostaglandins antibiotics, renal agents, respiratory agents, sedatives, sex hormones, stimulants, sympathomimetics, systemic anti-infectives, tactolimuls, thrombolytic agents, thyroid agents, treatments for attention deficit disorder, uterine-active agents, vaccines, vasodilators, xanthines, or a mixture of any two or more thereof.

21. The pharmaceutical formulation of claim 1, wherein the active pharmaceutical ingredient is fenofibrate, cyclosporine, sirolimus, danazol, naproxen, sildenafil, griseofulvin, mycophenolate mofetil, or a mixture of any two or more thereof.

22. The pharmaceutical formulation of claim 1, wherein the solvent is an alcohol, N-methylpyrrolidinone, methoxypolyethylene glycol, polyethylene glycol, polyethylene oxide, ethoxy diglycol, triacetin, dimethyl sulfoxide, propylene glycol, isopropyl myristate, mono-, di- or tri-glycerides, or a mixture of any two or more thereof.

23. The pharmaceutical formulation of claim 22, wherein the alcohol is benzyl alcohol, ethyl alcohol, or a mixture of any two or more thereof.

24. The pharmaceutical formulation of claim 22, wherein the polyethylene glycol has an average molecular weight of about 1000 g/mol or greater, and the methoxypolyethylene glycol has an average molecular weight of about 1000 g/mol or greater.

25. The pharmaceutical formulation of claim 22, wherein the polyethylene glycol has an average molecular weight of from about 1000 g/mol to about 20,000 g/mol, and the methoxypolyethylene glycol has an average molecular weight of from about 1000 g/mol to about 20,000 g/mol.

26. The pharmaceutical formulation of claim 1, wherein the non-miscible liquid is a fatty acid, a medium chain glyceride, a long chain glyceride, an ethyl ester of a fatty acid, a propylene glycol fatty acid ester, a sorbitan fatty acid ester, a polyglyceryl fatty acid ester, a glyceryl mono-, di-, or tri-caprylic acid ester; a glyceryl mono-, di-, or tri-capric acid esters; or a mixture of any two or more thereof.

27. The pharmaceutical formulation of claim 1, wherein the non-miscible liquid is selected from vegetable oils, nut oils, fish oils, lard oil, mineral oils, squalane, tricaprylin (1,2,3-trioctanoyl glycerol), and mixtures of any two or more thereof.

28. The pharmaceutical formulation of claim 27, wherein the non-miscible liquid is almond oil (sweet), apricot seed oil, borage oil, canola oil, coconut oil, corn oil, cotton seed oil, fish oil, jojoba bean oil, lard oil, linseed oil (boiled), macadamia nut oil, medium chain triglycerides, mineral oil, olive oil, origanum oil, peanut oil, safflower oil, sesame oil, soybean oil, sunflower seed oil, wheat germ oil, mineral oil (light), DL-α-tocopherol, ethyl oleate, ethyl linoleate, glyceryl behenate, glyceryl monooleate, glyceryl monostearate, glyceryl palmitostearate, linoleic acid, linolenic acid, oleic acid, palmitostearic acid, peppermint oil, polyglyceryl oleate, propylene glycol monolaureate, propylene glycol dilaureate, sorbitan monolaurate, sorbitan monooleate, sorbitan monopalmitate, sorbitan monostearate, sorbitan trioleate, stearic acid, tetraglyceryl monooleate, or a mixture of any two or more thereof.

29. The pharmaceutical formulation of claim 1, wherein the stabilizer is selected from non-phospholipid surfactants, non-phenol polyethylene glycol ethers, sorbitan esters, polyethylene glycol esters, block polymers, acrylic polymers, ethoxylated fatty acids, ethoxylated alcohols, ethoxylated fatty acid esters, monoglycerides, silicon-based surfactants, polysorbates, tergitols, sugar fatty acid ester; a sucrose mono-, di-, or tri-fatty acid ester; a polyoxyethylene castor oil compound; a polyoxyethylene sorbitan fatty acid ester; a polyoxyethylene mono- or di-fatty acid ester; a polyoxyethylene alkyl ether; a glyceryl mono-, di-, or tri-fatty acid ester; a mixtures of polyoxyethylene mono- or di-ester of a C₈-C₂₂ fatty acid; a glyceryl mono-, di-, or tri-ester of a C₈-C₂₂ fatty acid, or a mixture of any two or more thereof

30. The pharmaceutical formulation of claim 29, wherein the stabilizer is selected from ARLACEL™, BRIJ™, Cremophore RH-40, glycerin monostearate, PEMULEN™, PLURONIC™, polyethylene glycol stearate, polyoxyl 35 castor oil, polyoxyl 40 hydrogenated castor oil, polyoxyl 60 hydrogenated castor oil, polysorbate 20, polysorbate 40, polysorbate 60, polysorbate 80, polyoxyl 40 stearate, polyoxyl 40 oleate, polyoxyl 20 cetostearyl ether, polyoxyl 10 oleyl ether, sodium dioctyl sulfosuccinate, sodium lauryl sulfate, SPAN™, TERGITOL™ NP-40, TERGITOL™ NP-70, DL-α-tocopheryl polyethylene glycol succinate, TWEEN™ 20, TWEEN™ 60, TWEEN™ 80, or a mixture of any two or more thereof.

31. The pharmaceutical formulation of claim 1, wherein the multi-phasic pharmaceutical composition comprises globules of the non-miscible liquid and the globules have a diameter of less than about 10 μm.

32. The pharmaceutical formulation of claim 31, wherein the globules have a diameter of less than about 9 microns, less than about 8 microns, less than about 7 microns, less than about 6 microns, less than about 5 microns, less than about 4 microns, less than about 3 microns, less than about 2 microns, less than about 1000 nm, less than about 900 nm, less than about 800 nm, less than about 700 nm, less than about 600 nm, less than about 500 nm, less than about 400 nm, less than about 300 nm, less than about 290 nm, less than about 280 nm, less than about 270 nm, less than about 260 nm, less than about 250 nm, less than about 240 nm, less than about 230 nm, less than about 220 nm, less than about 210 nm, less than about 200 nm, less than about 190 nm, less than about 180 nm, less than about 170 nm, less than about 160 nm, less than about 150 nm, less than about 140 nm, less than about 130 nm, less than about 120 nm, less than about 110 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm.

33. The pharmaceutical formulation of claim 1, wherein an average diameter of the particles of the particulate state is less than about 1 micron.

34. The pharmaceutical formulation of claim 33, wherein the average diameter is less than about 900 nm, less than about 800 nm, less than about 700 nm, less than about 600 nm, less than about 500 nm, less than about 400 nm, less than about 300 nm, less than about 290 nm, less than about 280 nm, less than about 270 nm, less than about 260 nm, less than about 250 nm, less than about 240 nm, less than about 230 nm, less than about 220 nm, less than about 210 nm, less than about 200 nm, less than about 190 nm, less than about 180 nm, less than about 170 nm, less than about 160 nm, less than about 150 nm, less than about 140 nm, less than about 130 nm, less than about 120 nm, less than about 110 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm.

35. The pharmaceutical formulation of claim 1, further comprising an antioxidant, a coloring agent, a flavoring agent, a preservative, a sweetener, a volatile oil, or a mixture of any two or more thereof.

36. A method of preparing the pharmaceutical formulation of claim 1 comprising:
(a) mixing the active pharmaceutical ingredient, the solvent, the stabilizer, and the non-miscible liquid to form a first mixture;
(b) emulsifying the first mixture with water to form the multi-phasic pharmaceutical composition; and
(c) formulating the multi-phasic pharmaceutical composition as an oral dosage form.

37. The method of claim 36, wherein the oral dosage form is a liquid dosage form or a solid dosage form.

38. The method of claim 37, wherein when the oral dosage is a solid dosage form, the method further comprises:
(d) mixing the emulsified first mixture with an adsorbent carrier.
